(19) United States
(12) Patent Application Publication
(10) Pub. No.: US 2008/0145430 A1
(43) Pub. Date: Jun. 19, 2008

(54) Ophthalmic Nanoparticulate Formulation Of A Cyclooxygenase-2 Selective Inhibitor

(76) Inventors: Santipharp Panmai, North Wales, PA (US); Laman L. Alani, Lansdale, PA (US)

Correspondence Address: MERCK AND CO., INC, P O BOX 2000, RAHWAY, NJ, 07065-0907, US

(21) Appl. No.: 11/792,024

(22) PCT Filed: Dec. 5, 2005
(86) PCT No.: PCT/US05/43858

§ 371 Date: May 30, 2007

Related U.S. Application Data
(60) Provisional application No. 60/634,305, filed on Dec. 8, 2004.

(51) Int. Cl. A61K 9/10; A61K 31/365; A61K 31/444; A61K 31/415; A61K 31/42; A61K 31/196; A61P 27/02

(52) U.S. Cl. 424/489; 514/473; 514/334; 514/406; 514/378; 514/567

(57) ABSTRACT

The present invention is directed to a novel pharmaceutical composition suitable for ophthalmic use comprising nanoparticles dispersed in a liquid dispersion medium, wherein said nanoparticles comprise a cyclooxygenase-2 selective inhibitor having adsorbed on the surface thereof at least one surface stabilizer. The ophthalmic formulation of the present invention is stable, non-irritating and sufficiently bioavailable. The invention also encompasses a process for making as well as a kit for preparing the novel ophthalmic formulation.

### BACKGROUND OF THE INVENTION

Selective inhibitors of cyclooxygenase-2 are a sub-class of the class of drugs known as non-steroidal antiinflammatory drugs (NSAIDs). The NSAIDs are active in reducing the prostaglandin-induced pain and swelling associated with the inflammation process but are also active in affecting other prostaglandin-regulated processes not associated with the inflammation process. Thus, use of high doses of most common NSAIDs can produce severe side effects, including life threatening ulcers, that limit their therapeutic potential. An alternative to NSAIDs is the use of corticosteroids, which have even more drastic side effects, especially when long term therapy is involved.

Previous NSAIDs have been found to prevent the production of prostaglandin by inhibiting enzymes in the human arachidonic acid/prostaglandin pathway including the enzyme cyclooxygenase (COX). The discovery that there are two isoforms of the COX enzyme, the first, COX-1, being involved with physiological functions and the second, COX-2, being induced in inflamed tissue, has given rise to a new approach. While conventional NSAIDs block both forms of the enzyme, the identification of the inducible COX-2 enzyme associated with inflammation has provided a viable target of inhibition which more effectively reduces inflammation and produces fewer and less drastic side effects. Many compounds which have activity as COX-2 inhibitors have been identified, including rofecoxib (VIOXX®), etoricoxib (ARCOXIA™), celecoxib (CELEBREX®) and valdecoxib (BEXTRA™), and much research continues in this area.

The present invention is directed to a novel topical nanoparticulate pharmaceutical composition comprising a cyclooxygenase-2 selective inhibitor for treating a variety of ocular diseases or conditions. Many agents with activity as cyclooxygenase-2 inhibitors are poorly soluble and therefore are not easily adaptable to an ophthalmic formulation. Nanoparticulate compositions, first described in U.S. Pat. No. 5,145,684, are particles consisting of a poorly soluble therapeutic or diagnostic agents having adsorbed onto the surface thereof a non-crosslinked surface stabilizer. The present invention provides for an ophthalmic nanoparticulate pharmaceutical composition comprising a cyclooxygenase-2 selective inhibitor, which is stable, non-irritating and sufficiently bioavailable.

### SUMMARY OF THE INVENTION

The present invention is directed to a novel pharmaceutical composition suitable for ophthalmic use comprising nanoparticles dispersed in a liquid dispersion medium, wherein said nanoparticles comprise a cyclooxygenase-2 selective inhibitor having adsorbed on the surface thereof at least one surface stabilizer. The ophthalmic formulation of the present invention is stable, non-irritating and sufficiently bioavailable. The invention also encompasses a process for making as well as a kit for preparing the novel ophthalmic formulation.

### DESCRIPTION OF THE INVENTION

The invention encompasses a pharmaceutical composition suitable for ophthalmic use comprising nanoparticles dispersed in a liquid dispersion medium, wherein said nanoparticles comprise a cyclooxygenase-2 selective inhibitor having adsorbed on the surface thereof at least one surface stabilizer in a concentration sufficient to maintain a particle size distribution of:

(a) a mean particle size of less than about 200 nm; and

(b) 90% of the particles with a particle size of less than about 300 nm,

for at least a four week period at ambient temperature.

The term “ambient temperature” means about room temperature, i.e., 20° C. to 25° C.

An embodiment of the invention encompasses the pharmaceutical composition described above wherein the cyclooxygenase-2 selective inhibitor is selected from the group consisting of: rofecoxib, etoricoxib, celecoxib, valdecoxib and lumiracoxib.

Another embodiment of the invention encompasses the pharmaceutical composition described above wherein the cyclooxygenase-2 selective inhibitor is rofecoxib.

Another embodiment of the invention encompasses the pharmaceutical composition described above wherein the cyclooxygenase-2 selective inhibitor is etoricoxib.

Another embodiment of the invention encompasses the pharmaceutical composition described above wherein the cyclooxygenase-2 selective inhibitor is celecoxib.

Another embodiment of the invention encompasses the pharmaceutical composition described above wherein the surface stabilizer is selected from the group consisting of: hydroxypropyl cellulose, hydroxypropyl cellulose super low viscosity, hydroxypropyl cellulose-low viscosity, hydroxypropyl methyl cellulose, methyl cellulose, hydroxyethyl cellulose and carboxymethyl cellulose sodium. Within this embodiment the invention encompasses the above pharmaceutical composition wherein the surface stabilizers are selected from the group consisting of: (1) hydroxypropylmethyl cellulose K3 and POLOXAMER 407; and (2) hydroxypropylmethyl cellulose E3 and POLOXAMER 407. Also within this embodiment, the surface stabilizers are hydroxypropylmethyl cellulose K3 and POLOXAMER 407, wherein hydroxypropylmethylcellulose K3 is present in a concentration of about 10 mg/mL and POLOXAMER 407 is present in a concentration of about 0.5 mg/mL. Also within this embodiment the invention encompasses the above pharmaceutical composition wherein the surface stabilizers are hydroxypropylmethyl cellulose E3 and POLOXAMER 407, wherein hydroxypropylmethylcellulose E3 is present in a concentration of about 10 mg/mL and POLOXAMER 407 is present in a concentration of about 1 to 2 mg/mL. All concentrations expressed are with respect to the liquid dispersion medium.

Another embodiment of the invention encompasses the pharmaceutical composition described above wherein the cyclooxygenase-2 selective inhibitor is rofecoxib present in a concentration of about 50 mg/mL.

Another embodiment of the invention encompasses the pharmaceutical composition described above having an initial particle size distribution of:

(a) a mean particle size of less than about 100 nm; and

(b) 90% of the particles with a particle size of less than about 150 nm.

The term “initial particle size distribution” means the mean particle size and 90% of the particles values at the zero timepoint. See Example 2.

Another embodiment of the invention encompasses the pharmaceutical composition described above wherein the liquid dispersion medium is water.

Another embodiment of the invention encompasses the pharmaceutical composition described above wherein the liquid dispersion medium is an isotonic agent. Within this embodiment the invention encompasses the above pharmaceutical composition wherein the isotonic agent is selected from the group consisting of: NaCl (aq), such as 0.9% NaCl, and sugar (aq), such as 5% sugar. Sugar means, for example, sucrose, glucose, fructose and the like.

Another embodiment of the invention encompasses a kit to prepare the pharmaceutical composition described above comprising lyophilized nanoparticles, wherein said nanoparticles comprise a cycloxygenase-2 selective inhibitor having adsorbed on the surface thereof at least one surface stabilizer, said particles capable of being reconstituted in a liquid dispersion medium to prepare the pharmaceutical composition suitable for ophthalmic use, and wherein said surface stabilizer is present in a concentration sufficient to, upon reconstitution, maintain a particle size distribution of:

(a) a mean particle size of less than about 200 nm; and

(b) 90% of the particles with a particle size of less than about 300 nm, for at least a four week period at ambient temperature. Within this embodiment the invention encompasses the above kit wherein the liquid dispersion medium is water. Also within this embodiment the invention encompasses the above kit further comprising the liquid dispersion medium in a compartment separate from the lyophilized nanoparticles to be reconstituted with the lyophilized nanoparticles to prepare the pharmaceutical composition suitable for ophthalmic use.

The invention also encompasses a process for making a pharmaceutical composition suitable for ophthalmic use comprising nanoparticles dispersed in a liquid dispersion medium, wherein said nanoparticles comprise a cycloxygenase-2 selective inhibitor having adsorbed on the surface thereof at least one surface stabilizer, said process comprising:

(a) dispersing the cyclooxygenase-2 selective inhibitor and mixing at least one surface stabilizer in a liquid dispersion medium, and

(b) wet grinding the particles in the presence of rigid grinding media having an average particle size of about 500 μm, to make the pharmaceutical composition suitable for ophthalmic use comprising nanoparticles dispersed in a liquid dispersion medium, wherein said nanoparticles comprise a cyclooxygenase-2 selective inhibitor having adsorbed on the surface thereof at least one surface stabilizer in a concentration sufficient to maintain a particle size distribution of:

(a) a mean particle size of less than about 200 nm; and

(b) 90% of the particles with a particle size of less than about 300 nm, for at least a four week period at ambient temperature.

An embodiment of the invention encompasses the above process wherein the cyclooxygenase-2 selective inhibitor is selected from the group consisting of: rofecoxib, etoricoxib, celecoxib, valdecoxib and lumiracoxib.

Another embodiment of the invention encompasses the above process wherein the cyclooxygenase-2 selective inhibitor is rofecoxib.

Another embodiment of the invention encompasses the above process wherein the cyclooxygenase-2 selective inhibitor is etoricoxib.

Another embodiment of the invention encompasses the above process wherein the particles are sterilized by sterile filtration.

The invention also encompasses a pharmaceutical composition suitable for ophthalmic use comprising nanoparticles dispersed in a liquid dispersion medium, wherein said nanoparticles comprise a cyclooxygenase-2 selective inhibitor having adsorbed on the surface thereof at least one surface stabilizer, made by the process described above.

With respect to the indicated particle sizes, the term “about” means ±50 nm.

With respect to the indicated concentrations, the term “about” means ±0.2 mg/mL.

The invention also encompasses a method for treating an ocular cyclooxygenase-2 mediated disease or condition in a patient in need thereof comprising topically administering to the patient a therapeutically effective amount of the pharmaceutical composition described above. Within this embodiment, the cyclooxygenase-2 mediated disease or condition is selected from the group consisting of: post-operative inflammation and pain from ophthalmic surgery, retinitis, conjunctivitis, uveitis, ocular photophobia, acute injury to the eye tissue, corneal graft rejection, ocular neovascularization, retinal neovascularization, diabetic retinopathy, hypertensive retinopathy, macular degeneration, retinal, fibroplasias, glaucoma, blepharitis, post-operative inflammation and pain from corneal transplant surgery, endophthalmitis, episcleritis, keratitis, keratoconjunctivitis, keratoconjunctivitis sicca, Mooren's ulcer, macular edema, intraoperative miosis, ocular pain photophobia and sarcoidosis. Also within this embodiment, the cyclooxygenase-2 mediated disease or condition is selected from the group consisting of: uveitis, macular degeneration and diabetic retinopathy.

The invention also encompasses the above method further comprising concomitantly or sequentially administering ranibizumab in an amount effective together with a cyclooxygenase-2 selective inhibitor to treat macular degeneration.

The terms “inhibitor of cyclooxygenase-2”, “cyclooxygenase-2 selective inhibitor” and “COX-2 inhibitor” as used herein embrace compounds which selectively inhibit cyclooxygenase-2 over cyclooxygenase-1, including pharmaceutically acceptable salts thereof. Employing the human whole blood COX-1 assay and the human whole blood COX-2 assay described in C. Brideau et al, Inflamm. Res. 45: 68-74 (1996), herein incorporated by reference, preferably, the compounds have a cyclooxygenase-2 IC₅₀ of less than about 2 μM in the human whole blood COX-2 assay, yet have a cyclooxygenase-1 IC₅₀ of greater than about 5 μM in the human whole blood COX-1 assay. Also preferably, the compounds have a selectivity ratio of cyclooxygenase-2 inhibition over cyclooxygenase-1 inhibition of at least 10, and more preferably of at least 40. The resulting selectivity may indicate an ability to reduce the incidence of common NSAID-induced side effects, especially erosions and ulceration of the upper gastrointestinal mucosa.

Rofecoxib is known in the art (VIOXX, Merck & Co., Inc.). Rofecoxib is described as Example 23 in U.S. Pat. No. 5,474,995, granted Dec. 12, 1995. Methods for making rofecoxib are described in U.S. Pat. No. 5,840,924, granted Nov. 24, 1998.

Etoricoxib is known in the art and commercially available (ARCOXIA, Merck & Co., Inc.). Etoricoxib is described as Example 23 in U.S. Pat. No. 5,861,419, granted Jan. 19, 1999. Methods for making etoricoxib are described in U.S. Pat. No. 6,040,319, granted Mar. 21, 2000.

Celecoxib is described in the art and commercially available (CELEBREX, Pfizer, Inc.). Celecoxib is described in U.S. Pat. Nos. 5,466,823, 5,563,165, 5,760,068 and 5,972,986.

The present invention is useful to treat ocular diseases or conditions mediated by the cyclooxygenase-2 enzyme via topical administration to the eye. For example, the present invention would be useful to treat post-operative inflammation from ophthalmic surgery such as cataract surgery and refractive surgery. The invention would also be useful to treat ophthalmic disease such as retinitis, conjunctivitis, retinopathies, uveitis, ocular photophobia, and of the acute injury to the eye tissue. The invention would also be useful to treat ocular angiogenesis disorders, for example, corneal graft rejection, ocular neovascularization, retinal neovascularization including neovascularization following injury or infection, diabetic retinopathy, macular degeneration, retinal fibroplasias and neovascular glaucoma. The invention would also be useful to treat blepharitis, post-operative inflammation and pain from corneal transplant surgery, endophthalmitis, episcleritis, scleritis, keratitis, keratoconjunctivitis, keratoconjunctivitis sicca, post-operative inflammation and pain from lens implantation surgery, Mooren's ulcer and post operative inflammation and pain from retinal detachment surgery. In addition, the invention would be useful to treat glaucoma, macular edema, intraoperative miosis, ocular pain photophobia and sarcoidosis.

Types of retinopathies treated or prevented by the invention include, but are not limited to, hypertensive retinopathy and diabetic retinopathy. Types of macular edema treated or prevented by the methods of the invention include, but are not limited to, cystoid macular edema and macular edema associated with diabetic retinopathy. Ocular pain and ocular inflammation may be treated or prevented by the invention. Ocular pain and ocular inflammation treated or prevented by the methods of the invention may be related to acute or chronic injury to the eye tissue.

COX-2 mediated disorders of the eye for which the pharmaceutical compositions of the invention are useful include without limitation inflammatory disorders such as endophthalmitis, episcleritis, retinitis, iriditis, cyclitis, choroiditis, keratitis, conjunctivitis and blepharitis, including inflammation of more than one part of the eye, e.g., retinochoroiditis, iridocyclitis, iridocyclochoroiditis (also known as uveitis), keratoconjunctivitis, blepharoconjunctivitis, etc.; other COX-2 mediated retinopathies including diabetic retinopathy; ocular tumors; ocular photophobia; acute trauma of any tissue of the eye including postsurgical trauma, e.g., following cataract or corneal transplant surgery; postsurgical ocular inflammation; intraoperative miosis; corneal graft rejection; ocular, for example retinal, neovascularization including that following injury or infection; macular degeneration; cystoid macular edema; retrolental fibroplasia; neovascular glaucoma; ocular pain; and COX-2 mediated side effects from ocular 12 prostaglandin therapy including increased iridial pigmentation, disruption of the blood aqueous barrier and cystoid macular edema.

The term “treating” as used herein encompasses not only treating a patient to relieve the patient of the signs and symptoms of the disease or condition but also prophylactically treating an asymptomatic patient to prevent the onset or progression of the disease or condition.

The term “concomitantly administering” means administering the agents substantially concurrently. The term “concomitantly administering” encompasses not only administering the two agents in a single pharmaceutical dosage form but also the administration of each active agent in its own separate pharmaceutical dosage formulation. Where separate dosage formulations are used, the agents can be administered at essentially the same time, i.e., concurrently.

The term “sequentially administering” means administering the agents at separately staggered times. Thus, agents can be sequentially administered such that the beneficial pharmaceutical effect of each agent is realized by the patient at substantially the same time. Thus, for example, if two active agents are both administered on a once a day basis, the interval of separation between sequential administration of the two agents can be up to twelve hours apart.

The present invention can be used as co-therapy with a vascular endothelial cell growth factor (VEGF) antagonist, including monoclonal antibodies, for treating age-related macular degeneration. Endothelial cell growth antagonists are disclosed, for example, in WO 96/30046, published Oct. 3, 1996, WO 00/37502, published Jun. 29, 2000, U.S. Pub. No. 2001/0021382, published Sep. 13, 2001, U.S. Pub. No. 2002/0032313, published Mar. 14, 2002, U.S. Pub. No. 2002/0098187, published Jul. 25, 2002, U.S. Pub. No. 2002/0122797, published Sep. 5, 2002, U.S. Pub. No. 2003/0023046, published Jan. 30, 2003, U.S. Pub. No. 2003/0203409, published Oct. 30, 2003, U.S. Pub. No. 2003/0206899, published Nov. 6, 2003, and U.S. Pat. No. 6,582,959, granted Jun. 24, 2003, all of which are hereby incorporated by reference in their entirety. In particular, the present invention can be used a co-therapy with LUCENTIS (ranibizumab), a humanized, therapeutic antibody fragment that is designed to bind and inhibit VEGF, for the treatment of age-related macular degeneration. For purposes of this co-therapy, ranibizumab may be administered by intravitreal injection at a dose between 0.1 mg to 1.0 mg. In addition, ranibixumab may also be administered via sub-tenons injection (using a solution, suspension, ointment, or sustained-release device), intra-scleral injection (using a solution, suspension, ointment, or sustained-release device), trans-scleral injection (using a solution, suspension, ointment, or sustained-release device), sub-conjunctival injection (using a solution, suspension, ointment, or sustained-release device), topical (using a solution, suspension, ointment, or sustained-release device), oral, intramuscular, subcutaneous, or transdermal (using a solution, suspension, ointment, or sustained-release device for any route).

The compound having at least one surface stabilizer adsorbed on the surface thereof to maintain an effective average particle size as set forth herein is also referred to herein as the active ingredient “nanoparticles” or “nanoparticulate drug particles.”

The invention provides a pharmaceutical composition comprising a liquid dispersion medium and the above-described nanoparticles dispersed therein. The terms “dispersion” or “suspension” are synonymous and used interchangeably herein and refer to a formulation in which the active ingredient nanoparticles remain suspended undissolved in a fluid such as water.

The present invention is further directed to methods of treatment comprising administering to a patient in need thereof a therapeutically effective amount of a pharmaceutical composition according to the present invention.

The nanoparticulate compositions of the invention have an effective average particle size of less as set forth herein. Surface stabilizers useful herein physically adhere to the surface of the compound, but do not chemically react with the drug or itself. Individually adsorbed molecules of the surface stabilizer are essentially free of intermolecular cross-linkages.

The present invention also includes nanoparticulate compositions having at least one surface stabilizer adsorbed on the surface thereof, formulated into compositions together with one or more non-toxic physiologically acceptable carriers, adjuvants, or vehicles, collectively referred to as carriers. The compositions are formulated for topical administration to the eye.

Useful surface stabilizers, which are known in the art and described for example in U.S. Pat. No. 5,145,684, are believed to include those which physically adhere to the surface of the active agent but do not chemically bond to or interact with the active agent. The surface stabilizer is adsorbed on the surface of the cyclooxygenase-2 selective inhibitor in a concentration sufficient to maintain an effective average particle size of less as set forth herein for the active agent. Furthermore, the individually adsorbed molecules of the surface stabilizer are essentially free of intermolecular cross-linkages. Two or more surface stabilizers can be employed in the compositions and methods of the invention. The surface stabilizers employed in the present invention must also be suitable for use in topical administration to the eye.

Suitable surface stabilizers can preferably be selected from known organic and inorganic pharmaceutical excipients. Such excipients include various polymers, low molecular weight oligomers, natural products, and surfactants. Preferred surface stabilizers include nonionic and ionic surfactants.

Representative examples of surface stabilizers include gelatin, casein, lecithin (phosphatides), dextran, gum acacia, cholesterol, tragacanth, stearic acid, benzalkonium chloride, calcium stearate, glycerol monostearate, cetostearyl alcohol, cetomacrogol emulsifying wax, sorbitan esters, polyoxyethylene alkyl ethers (e.g., macrogol ethers such as cetomacrogol 1000), polyoxyethylene castor oil derivatives, polyoxyethylene sorbitan fatty acid esters (e.g., the commercially available Tweens® such as e.g., Tween 20® and Tween 80® (ICI Speciality Chemicals)); polyethylene glycols (e.g., Carbowaxs 3550® and 934® (Union Carbide)), polyoxyethylene stearates, colloidal silicon dioxide, phosphates, sodium dodecylsulfate, carboxymethylcellulose calcium, carboxymethylcellulose sodium, methylcellulose, hydroxyethylcellulose, hydroxypropylcellulose, hydroxypropylmethyl-cellulose, hydroxypropylmethyl-cellulose phthalate, noncrystalline cellulose, magnesium aluminium silicate, triethanolamine, polyvinyl alcohol (PVA), polyvinylpyrrolidone (PVP), 4-(1,1,3,3-tetramethylbutyl)-phenol polymer with ethylene oxide and formaldehyde (also known as tyloxapol, superione, and triton), poloxamers (e.g., Pluronics F68® and F108®, which are block copolymers of ethylene oxide and propylene oxide); poloxamines (e.g., Tetronic 908®, also known as Poloxamine 908®, which is a tetrafunctional block copolymer derived from sequential addition of propylene oxide and ethylene oxide to ethylenediamine (BASF Wyandotte Corporation, Parsippany, N.J.)); Tetronic 15080® (T-1508) (BASF Wyandotte Corporation), dialkylesters of sodium sulfosuccinic acid (e.g., Aerosol OT®, which is a dioctyl ester of sodium sulfosuccinic acid (American Cyanamid)), dioctyl sodium sulfosuccinate (DOSS), docusate Sodium (Ashland Chem. Co., Columbus, Ohio); Duponol P®, which is a sodium lauryl sulfate (DuPont); Tritons X-200®, which is an alkyl aryl polyether sulfonate (Rohm and Haas); Crodestas F-110®, which is a mixture of sucrose stearate and sucrose distearate (Croda Inc.); p-isononylphenoxy-poly-(glycidol), also known as Olin-lOG® or Surfactant 10-G® (Olin Chemicals, Stamford, Conn.); Crodestas SL-40® (Croda, Inc.); and SA9OHCO, which is C₁₈H₃₇CH₂(CON(CH₃)—CH₂(CHOH)₄(CH₂0H)₂ (Eastman Kodak Co.); decanoyl-N-methylglucamide; n-decyl β-D-glucopyranoside; n-decyl β-D-maltopyranoside; n-dodecyl β-D-glucopyranoside; n-dodecyl β-D-maltoside; heptanoyl-N-methyl-glucamide; n-heptyl-β-D-glucopyranoside; n-heptyl β-D-thioglucoside; n-hexyl β-D-glucopyranoside; nonanoyl-N-methylglucamide; n-noyl β-D-glucopyranoside; octanoyl-N-methylglucamide; n-octyl-β-D-glucopyranoside; octyl β-D-thioglucopyranoside; and the like.

Most of these surface stabilizers are known pharmaceutical excipients and are described in detail in the Handbook of Pharmaceutical Excipients, published jointly by the American Pharmaceutical Association and The Pharmaceutical Society of Great Britain (The Pharmaceutical Press, 1986), specifically incorporated by reference. The surface stabilizers are commercially available and/or can be prepared by techniques known in the art.

The nanoparticles of the invention contain a discrete phase of an active ingredient with the surface stabilizer adsorbed on the surface thereof. The surface stabilizer physically adheres to the active ingredient, but it does not chemically bond to or chemically react with the drug. Such chemical bonding or interaction would be undesirable as it could result in altering the function of the drug. The surface stabilizer is adsorbed on the surface of the active ingredient in a concentration sufficient to maintain an effective average particle size of as set forth herein. Furthermore, the individually adsorbed molecules of the surface stabilizer are essentially free of intermolecular cross-linkages.

In an aspect of the present invention the surface stabilizer is selected from hydroxypropyl cellulose (HPC), which is an ether of cellulose, HPC super low viscosity (HPC-SL), HPC-low viscosity (HPC-L), and hydroxypropyl methyl cellulose (HPMC). Preferred surface stabilizers include, but are not limited to hydroxypropyl cellulose (HPC), HPC-SL, HPC-L, methyl cellulose, hydroxyethyl cellulose, carboxymethyl cellulose sodium or hydroxypropyl methylcellulose (HPMC). (see, e.g., Remington's at pp. 1304-1308). Preferably, HPC, HPC-L or HPMC are used as surface stabilizers; HPC-SL may also be used as a surface stabilizer.

The relative amount of the compound active ingredients and one or more surface stabilizers can vary widely. The optimal amount of the surface stabilizers can depend, for example, upon hydrophilic lipophilic balance (HLB), melting point, and water solubility of the surface stabilizer, and the surface tension of water solutions of the stabilizer, etc.

As used herein, particle size is determined on the basis of the weight average particle size as measured by conventional particle size measuring techniques well known to those skilled in the art. Such techniques include, for example, sedimentation field flow fractionation, photon correlation spectroscopy, light scattering, and disk centrifugation.

The terms “dispersion” and “suspension” are synonymous and used interchangeably herein and refer to a formulation where the ingredient particles remain suspended undissolved in a fluid such as water.

The term “patient” or “subject” as used herein refers to an animal, preferably a mammal, most preferably a human (such as an adult, including an elderly adult such as an elderly man or an elderly woman), who has been the object of treatment, observation or experiment.

The term “therapeutically effective amount” as used herein means that amount of active compound or pharmaceutical agent that elicits the biological or medicinal response in a tissue, system, animal or human that is being sought by a researcher, veterinarian, medical doctor or other clinician, which includes alleviation of the symptoms of the disease being treated.

The phrase “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

The present invention provides for a pharmaceutical composition for ophthalmic use that is stable, non-irritating and sufficiently bioavailable.

The term “liquid dispersion medium” includes water, safflower oil, ethanol, t-butanol, hexane or glycol. Preferably, the liquid dispersion medium is water.

The nanoparticulate compositions can be made using, for example, milling or precipitation techniques. Exemplary methods of making nanoparticulate compositions are described in U.S. Pat. Nos. 5,145,684 and 5,862,999.

The nanoparticulate drug particles of the present invention can be prepared by first dispersing the cyclooxygenase-2 selective inhibitor in the presence of one or more surface stablizers in a liquid dispersion medium followed by applying mechanical means in the presence of grinding media to reduce the particle size of the active ingredient to an effective average particle size as set forth herein.

A general procedure for preparing the drug nanoparticles of the invention is set forth below. The active ingredient is either obtained commercially or prepared by techniques known in the art in a conventional coarse form. It is preferred, but not essential, that the particle size of the selected drug be less than about 100 μm as determined by sieve analysis. If the coarse particle size of the drug is greater than about 100 μm, then it is preferred that the drug particles be reduced in size to less than about 100 μm using a conventional milling method, such as airjet or fragmentation milling, prior to reducing the particulate drug to submicron particle size.

The coarse drug particles can then be added to a liquid medium in which the drug is essentially insoluble to form a premix. The concentration of the drug in the liquid medium can vary from about 0.1 to about 60%, but is preferably from about 5 to about 30% (w/w). It is preferred, but not essential, that the surface stabilizer is present in the premix. The concentration of the surface stabilizer can vary from about 0.1 to about 90%, but it is preferably from about 1 to about 75%, and more preferably from about 20 to about 60%, by weight based upon the total combined weight of the active ingredient and surface stabilizer. The apparent viscosity of the premix suspension is preferably less than about 1000 centipoise.

The premix can be used directly by subjecting it to mechanical means to reduce the average particle size in the dispersion as set forth herein. It is preferred that the premix be used directly when a ball mill is used for attrition. Alternatively, the active ingredient, and optionally the surface stabilizer, can be dispersed in the liquid medium using suitable agitation, such as a roller mill or a Cowles-type mixer, until a homogeneous dispersion is observed. In a homogeneous dispersion, no large agglomerates are visible to the naked eye. It is preferred that the premix be subjected to such a premilling dispersion step when a recirculating media mill is used for attrition.

The mechanical means applied to reduce the particle size of the active ingredient can be a dispersion mill. Suitable dispersion mills include, but are not limited to, a ball mill, an attritor mill, a vibratory mill, and media mills such as a sand mill or a bead mill. A media mill is preferred due to the relatively shorter milling time required to provide the desired reduction in particle size. For media milling, the apparent viscosity of the premix is preferably from about 100 to about 1000 centipoise. For ball milling, the apparent viscosity of the premix is preferably from about 1 to about 100 centipoise. Such ranges tend to afford an optimal balance between efficient particle fragmentation and media erosion.

The attrition time can vary widely and depends primarily upon the particular mechanical means and processing conditions selected. For ball mills, processing times of up to five days or longer may be required. Using a high shear media mill, processing times of less than 1 day (residence times of from one minute up to several hours) have provided the desired results.

The drug particles must be reduced in size at a temperature which does not significantly degrade the active ingredient. Processing temperatures of less than about 30-40° C. are ordinarily preferred. If desired, the processing equipment can be cooled with conventional cooling equipment. Generally, the methods of the invention can be conveniently carried out under conditions of ambient temperature and at processing pressures which are safe and effective for the milling process. For example, ambient processing pressures are typical of ball mills, attritor mills, and vibratory mills. Control of the temperature, for example, by jacketing or immersion of the milling chamber in ice water, is encompassed by the invention.

Processing pressures from about 1 psi (0.07 kg/cm2) up to about 50 psi (3.5 kg/cm2) are encompassed by the invention. Processing pressures typically range from about 10 psi to about 20 psi.

The surface stabilizer, if not present in the premix, must be added to the dispersion after attrition in an amount as described for the premix above. Thereafter, the dispersion can be mixed by, for example, shaking vigorously. Optionally, the dispersion can be subjected to a sonication step using, for example, an ultrasonic power supply. In such a method, the ultrasonic power supply can, for example, release ultrasonic energy having a frequency of about 20 to about 80 kHz for a time of about 1 to about 120 seconds.

After attrition is completed, the grinding media is separated from the milled particulate product using conventional separation techniques, such as by filtration, sieving through a mesh screen, and the like. The surface stabilizer is added to the milled particulate product either before or after the milled product is separated from the grinding media.

In a preferred grinding process, the particles are made continuously. In such a continuous method, the slurry of active ingredient/surface stabilizer and optionally an additional surface stablizer is continuously introduced into a milling chamber, the active ingredient is continuously contacted with grinding media while in the chamber to reduce the particle size of the active ingredient, and the active ingredient is continuously removed from the milling chamber. The surface stabilizer, either alone or in conjunction with one or more additional surface stabilizers, can also be continuously added to the media chamber along with the active ingredient, or it can be added to the active ingredient which is removed from the chamber following grinding.

The resulting dispersion of the present invention is stable and comprises the liquid dispersion medium described above. The dispersion of surface stabilizer and nanoparticulate active ingredient can be spray dried, spray coated onto a solid support such as cellulose spheres or sugar spheres or other pharmaceutical excipients using techniques well known in the art.

The grinding media for the particle size reduction step can be selected from rigid media which is preferably spherical or particulate in form and which has an average size of less than about 3 mm and, more preferably, less than about 1 mm. Such media can provide the desired drug particles of the invention with shorter processing times and impart less wear to the milling equipment. The selection of material for the grinding media is not believed to be critical. Zirconium oxide, such as 95% ZrO stabilized with yttrium and 95% ZrO stabilized with magnesia, zirconium silicate, and glass grinding media have been found to provide particles having acceptable minimal levels of contamination for the preparation of pharmaceutical compositions. Other media, such as stainless steel, titania, and alumina can also be used. Preferred grinding media have a density greater than about 3 g/cm³.

The grinding media can comprise particles, preferably spherical in shape, such as beads, consisting of essentially polymeric resin. Alternatively, the grinding media can comprise particles having a core with a coating of the polymeric resin adhered thereto. The media can range in size from about 0.1 to about 3 mm. For fine grinding, the particles preferably are from about 0.2 to about 2 mm, and more preferably, from about 0.25 to about 1 mm in size.

The polymeric resin can have a density from about 0.8 to about 3.0 g/cm³. Higher density resins are preferred as such resins can provide more efficient particle size reduction.

In general, polymeric resins suitable for use in the present invention are chemically and physically inert, substantially free of metals, solvent, and monomers, and of sufficient hardness and friability to enable them to avoid being chipped or crushed during grinding. Suitable polymeric resins include, but are not limited to, cross-linked polystyrenes, such as polystyrene cross-linked with divinylbenzene, styrene copolymers, polycarbonates, polyacetals such as Delrin®, vinyl chloride polymers and copolymers, polyurethanes, polyamides, poly(tetrafluoroethylenes), such as Teflon® and other fluoropolymers, high density polyethylenes, polypropylenes, cellulose ethers and esters such as cellulose acetate, polyhydroxymethacrylate, polyhydroxyethyl acrylate, silicone containing polymers such as polysiloxanes, and the like. The polymer can also be biodegradable. Exemplary biodegradabe polymers include, but are not limited to, poly(lactides), poly(glycolide) copolymers of lactides and glycolide, polyanhydrides, poly(hydroxy-ethyl methacylate), poly(imino carbonates), poly(N-acylhydroxyproline)esters, poly(N-palmitoyl hydroxyproline) esters, ethylene-vinyl acetate copolymers, poly(orthoesters), poly(caprolactones), and poly(phoshazenes). For biodegradable polymers, contamination of the resultant composition from the media itself can advantageously metabolize in vivo into biologically acceptable products that can be eliminated from the body.

The grinding media is separated from the milled particulate active ingredient using conventional separation techniques in a secondary process, such as by filtration, sieving through a mesh filter or screen, and the like. Other separation techniques such as centrifugation may also be employed.

As used herein, particle size is determined on the basis of the average particle size as measured by conventional techniques well known to those skilled in the art, such as sedimentation field flow fractionation, photon correlation spectroscopy, or disk centrifugation. When photon correlation spectroscopy (PCS) is used as the method of particle sizing, the average particle diameter is the Z-average particle diameter known to those skilled in the art.

The concentration of the one or more surface stabilizers can vary from about 0.01 to about 90%, from about 1 to about 75%, from about 10 to about 60%, or from about 10 to about 30% by weight based on the total combined dry weight of the drug substance and surface stabilizer. The concentration of the cyclooxygenase-2 selective inhibitor can vary from about 99.99% to about 10%, from about 99% to about 25%, from about 90% to about 40%, or from about 90% to about 70% by weight based on the total combined dry weight of the compound, the surface stabilizer and other excipients.

The surface stabilizer is preferably present in a concentration of about 0.1 to about 10 mg per square meter of surface area of the active ingredient, or in a concentration of about 0.1 to about 90%, and more preferably about 5 to about 50% by weight based upon the total weight of the dry particle. Alternatively, the surface stabilizer is present at a concentration of about 1-20% by weight, preferably about 2-15% by weight, and more preferably about 3-10% by weight.

The invention is exemplified by the following non-limiting examples.

### EXAMPLE 1

A nanosuspension of rofecoxib was prepared using the Nanomill (Model-01, manufactured by Elan). The selected stabilizers were hydroxymethylcellulose K3 (HPMC K3) (Dow Chemical) and Poloxamer 407 (Lutrol, BASF). The following ingredients were added to the 10-mL chamber: 5.6 grams of 500-micron polystyrene beads (Elan) and 4.60 grams of the slurry. The target concentration of the slurry is 5% wt drug, 1% wt HPMC K3, 0% to 0.1% wt Poloxamer P407. For a Poloxamer 407 concentration of 0.05% wt, 0.23 gram of drug was added to 4.37 grams of solution, which consisted of 0.046 gram of HPMC K3, 0.0023 gram of Poloxamer 407, and the remaining is water-for-injection (Abbott). The milling conditions were as follows: time=1 to 2 hr; speed=5500 rpm; temperature=5° C. After milling, the nanosuspension was separated from the media via filtration through a 100 μm mesh (Whatman) and stored under both refrigerated and ambient conditions.

### EXAMPLE 2

### Stability Test

The stability of the nanosuspensions was determined via particle size analysis. The particle size of the nanoparticles was measured using the Horiba LA-910 (Horiba Instruments, Inc.). The dispersing medium is water. A refractive index of 1.6 was assumed. The particle size of 5% wt drug, 1% wt HPMC K3 nanosuspensions with different concentrations of Poloxamer 407 concentration is shown in Table 1. The mean particle size values at the zero timepoint and after 4 weeks of storage at room temperature are reported. In this case, the Poloxamer 407 concentration of 0.05% wt resulted in the most stable nanosuspension, with sufficient physical stability after 4-week storage at RT.

TABLE 1
Formulation composition

| 5% wt Drug, | | Mean Particle Size | |
|---|---|---|---|
| 1% wt HPMC K3 | initial | 1 week at RT | 4 weeks at RT |
| +0.025% Poloxamer 407 | 103 nm | 18000 nm (3 days) | 217 nm |
| +0.05% Poloxamer 407 | 98 nm | 138 nm (3 days) | (28 days) |
| +0.1% Poloxamer 407 | 160 nm | 1130 nm (5 days) | |

### EXAMPLE 3

### Irritability Test

Ocular Administration. A nanosuspension of 5% wt drug, 1% wt HPMC K3, 0.05% wt Poloxamer 407 was prepared by following the protocol outlined in EXAMPLE 1. The mean particle size was around 100 nm. A control diluent containing 1% wt HPMC K3 and 0.05% wt Poloxame 407 was also prepared. For the tolerability study, three different drug concentrations were to be evaluated—5%, 0.5%, and 0.05% wt drug—plus the control diluent. To prepare 0.5% wt drug nanosuspension, 1 part of 5% wt drug nanosuspension was diluted with 9 parts of the control diluent. To prepare 0.05% wt drug nanosuspension, 1 part of 5% wt drug nanosuspension was diluted with 99 parts of the control diluent. New Zealand white (NZW), male rabbits, weighing 2.5 to 4 kg, were dosed topically and bilaterally with 25 μl vehicle of drug. Non-biomicroscopic, non-dilated examination of the ocular adnexa was performed by a trained operator to observe any potential irritation indicated by blinking, eye closure, lacrimation, chemosis hyperemia and general discomfort. These cage-side observations were made at the time of administration and at intervals of one hour post drug or vehicle instillation for up to six hours.

Tolerability results. All formulations were well-tolerated. There were no ocular adverse effects or drug-related findings.

### EXAMPLE 4

### Bioavailability

Ocular Administration. Dutch Belted rabbits of either sex, weighing 2.5 to 4 kg, were used in this study. The rabbits were administered eye drops bilaterally b.i.d. over a period of 10 days. Each drop is 25 μL. There were two groups of three rabbits each. One group received a nanosuspension of 5% wt drug, 1% wt HPMC K3, 0.05% wt Poloxamer 407, with a mean particle size of 100 nm (from EXAMPLE 1). The other group received the control diluent containing 1% wt HPMC K3, 0.05% Poloxamer 407. After the 10^{th} day, the eye tissues were harvested and stored frozen until drug extraction and analysis.

Extraction. The next step is the extraction of the drug sample. The eye tissue samples were homogenized with a 10-fold excess volume of 1:1 acetonitrile:water (the assumption is that 1 gram of tissue is equivalent to 1 mL). The PowerGen 125 homogenizer (Fisher Scientific) was used. ACN and HPLC water were purchased from Fisher Scientific. 100 μL of tissue homogenate was used per analysis. The drug in the tissue homogenates was extracted via liquid/liquid extraction with MTBE (Fisher Scientific).

Analysis. The last step is the analysis of the drug concentration. The liquid/liquid extracts were analyzed by LC/MS/MS. The API 3000 from Perkin-Elmer was used. The drug concentration was determined based on a calibration curve generated in the control tissue (the dynamic range is from 25 to 10,000 ng/gram).

Bioavailability results. The drug is bioavailable in the various parts of the eyes, when nanosuspension was administered. The drug concentrations in the various eye tissues are shown below. Specifically, the drug is present in the tissues of interest, e.g., the iris/ciliary body, retina, and choroid, at concentrations around 400-1000 ng/g. No drug was detected in any parts of the eyes, when administered with the diluent.

| | Drug concentration [ng/gram tissue] | | | | | | |
|---|---|---|---|---|---|---|---|
| | Rabbit #1 | | Rabbit #2 | | Rabbit #3 | | |
| | Left | Right | Left | Right | Left | Right | Average |
| Tissue | Eye | Eye | Eye | Eye | Eye | Eye | [ng/gram] |
| Aqueous | 271 | 294 | 289 | 197 | 156 | 301 | 251 ± 60 |
| Humor | | | | | | | |
| Vitreous | 9 | 21 | 17 | 3 | 8 | 13 | 12 ± 7 |
| Humor | | | | | | | |
| Cornea | 5930 | 7520 | 7330 | 6110 | 4880 | 7870 | 6610 ± 1150 |
| Sclera | 693 | 647 | 1950 | 3620 | 4030 | 11200 | 3690 ± 3944 |
| Ciliary | 1180 | 1320 | 1300 | 777 | 751 | 1080 | 1070 ± 251 |
| Body/Iris | | | | | | | |
| Retina | 125 | 90 | 182 | 186 | 1350 | 722 | 443 ± 502 |
| Choroid | 794 | 804 | 1100 | 772 | 1550 | 1340 | 1060 ± 328 |

### EXAMPLE 5

### Formulation and Process for Lyophilizing and Sterilization

Lyophilization+reconstitution. A nanosuspension containing 5% wt drug, 1% wt HPMC K3, 0.05% wt Poloxamer 407 was diluted with sugar (e.g., mannitol, dextrose, sucrose, and, lactose) in a 1:1 sugar:drug weight ratio. 1 part of sugar was added to 20 parts of the nanosuspension. The sugar-containing nanosuspension was frozen in a −70° C. freezer (Form a Scientific) and lyophilized using the Genesis 25XL lyophilizer (Virtis). The lyophilized cake was reconstituted with USP water. The mean particle size of the reconstituted nanosuspension, after sonication, is 105 nm.

Sterile filtration. A nanosuspension containing 5% wt drug, 1% wt HPMC K3, 0.05% wt Poloxamer 407 was filtered through a 0.2-micron Nylon filter (Whatman). The particle size of the filtrate was measured using the protocol described in Section (B). The mean particle size was 101 nm, and the D90 was 142 nm. The mean particle size of the filtrate remains smaller than 200 nm after 4 weeks.

While the invention has been described and illustrated with reference to certain particular embodiments thereof, those skilled in the art will appreciate that various adaptations, changes, modifications, substitutions, deletions, or additions of procedures and protocols may be made without departing from the spirit and scope of the invention.

The invention also encompasses a pharmaceutical composition suitable for ophthalmic use comprising nanoparticles dispersed in a liquid dispersion medium, wherein said nanoparticles comprise a cyclooxygenase-2 selective inhibitor having adsorbed on the surface thereof at least one surface stabilizer such that the pharmaceutical composition delivers a local drug concentration of the cyclooxygenase-2 inhibitor in the eye that is effective for the treatment of an ocular cyclooxygenase-2 mediated disease.

The invention also encompasses a pharmaceutical composition suitable for ophthalmic use comprising nanoparticles dispersed in a liquid dispersion medium, wherein said nanoparticles comprise a cyclooxygenase-2 selective inhibitor having adsorbed on the surface thereof at least one surface stabilizer in a concentration sufficient to maintain a particle size distribution of:

(a) a mean particle size of less than about 400 nm; and

(b) 90% of the particles with a particle size of less than about 500 nm,

for at least a four week period at ambient temperature.

In an embodiment of the invention, the surface stabilizer is selected from the group consisting of: hydroxypropyl cellulose, hydroxypropyl cellulose super low viscosity, hydroxypropyl cellulose-low viscosity, hydroxypropyl methyl cellulose, methyl cellulose, hydroxyethyl cellulose, carboxymethyl cellulose sodium, polyvinylpyrrolidone (PVP), polyvinyl alcohol (PVA), poloxamer (such as Poloxamer 188, Poloxamer 388, and Poloxamer 407), tyloxapol, polyoxyethylene sorbitan fatty acid esters (such as Tween 20 and Tween 80) and polyethylene glycol.

Within this embodiment, the surface stabilizers are selected from the group consisting of: hydroxypropylmethylcellulose and poloxamer. In a subset of this embodiment, hydroxypropylmethylcellulose is selected from HPMC E3 and HPMC K3, and poloxamer is POLOXAMER 407. The invention also encompasses pharmaceutical compositions of the invention wherein the surface stabilizers are hydroxypropylmethyl cellulose K3 and POLOXAMER 407, wherein hydroxypropylmethylcellulose K3 is present in a concentration of about 0.01 to about 200 mg/mL and POLOXAMER 407 is present in a concentration of about 0.001 to about 200 mg/mL; wherein hydroxypropylmethylcellulose K3 is present in a concentration of about 0.1 to about 100 mg/mL and POLOXAMER 407 is present in a concentration of about 0.01 to about 50 mg/mL; and wherein hydroxypropylmethylcellulose K3 is present in a concentration of about 1 to about 50 mg/mL and POLOXAMER 407 is present in a concentration of about 0.1 to about 10 mg/mL.

In another embodiment of the invention, the surface stabilizers are hydroxypropylmethyl cellulose E3 and POLOXAMER 407, wherein hydroxypropylmethylcellulose E3 is present in a concentration of about 0.01 to about 200 mg/mL and POLOXAMER 407 is present in a concentration of about 0.001 to about 200 mg/mL; wherein hydroxypropylmethylcellulose E3 is present in a concentration of about 0.1 to about 100 mg/mL and POLOXAMER 407 is present in a concentration of about 0.01 to about 50 mg/mL; and wherein hydroxypropylmethylcellulose E3 is present in a concentration of about 1 to about 50 mg/mL and POLOXAMER 407 is present in a concentration of about 0.1 to about 10 mg/mL.

In another embodiment of the invention the cyclooxygenase-2 selective inhibitor is rofecoxib present in a concentration of about 0.01 to about 600 mg/mL. Within this embodiment, the invention encompasses rofecoxib present in a concentration of about 1 to about 300 mg/mL. Also within this embodiment, the invention encompasses rofecoxib present in a concentration of about 10 to about 100 mg/mL.

The invention also encompasses pharmaceutical having an initial particle size distribution of: a mean particle size of less than about 400 nm and 90% of the particles with a particle size less than about 500 nm. Within this embodiment, the invention encompassed an initial particle size distribution of: a mean particle size of less than about 200 nm and 90% of the particles with a particle size less than about 300 nm.

The invention also encompasses a kit to prepare the pharmaceutical composition comprising lyophilized nanoparticles, wherein said nanoparticles comprise a cycloxygenase-2 selective inhibitor having adsorbed on the surface thereof at least one surface stabilizer, said particles capable of being reconstituted in a liquid dispersion medium to prepare the pharmaceutical composition suitable for ophthalmic use,

and wherein said at least one surface stabilizer is present in a concentration sufficient to, upon reconstitution, maintain a particle size distribution of: a mean particle size of less than about 400 nm, and 90% of the particles with a particle size less than about 500 nm, for at least a four week period at ambient temperature.

The invention also encompasses a process for making a pharmaceutical composition suitable for ophthalmic use comprising nanoparticles dispersed in a liquid dispersion medium, wherein said nanoparticles comprise a cycloxygenase-2 selective inhibitor having adsorbed on the surface thereof at least one surface stabilizer, said process comprising:

(a) dispersing the cyclooxygenase-2 selective inhibitor and mixing at least one surface stabilizer in a liquid dispersion medium, and

(b) wet grinding the particles in the presence of rigid grinding media having an average particle size of about 500 nm,

to make the pharmaceutical composition suitable for ophthalmic use comprising nanoparticles dispersed in a liquid dispersion medium, wherein said nanoparticles comprise a cyclooxygenase-2 selective inhibitor having adsorbed on the surface thereof at least one surface stabilizer in a concentration sufficient to maintain a particle size distribution of:

(a) a mean particle size of less than about 400 nm; and

(b) 90% of the particles with a particle size of less than about 500 nm,

for at least a four week period at ambient temperature.

1. A pharmaceutical composition suitable for ophthalmic use comprising nanoparticles dispersed in a liquid dispersion medium, wherein said nanoparticles comprise a cyclooxygenase-2 selective inhibitor having adsorbed on the surface thereof at least one surface stabilizer such that the pharmaceutical composition delivers a local drug concentration of the cyclooxygenase-2 inhibitor in the eye that is effective for the treatment of an ocular cyclooxygenase-2 mediated disease.

2. A pharmaceutical composition suitable for ophthalmic use in accordance with claim 1 comprising nanoparticles dispersed in a liquid dispersion medium, wherein said nanoparticles comprise a cyclooxygenase-2 selective inhibitor having adsorbed on the surface thereof at least one surface stabilizer in a concentration sufficient to maintain a particle size distribution of:
(a) a mean particle size of less than about 400 nm; and
(b) 90% of the particles with a particle size of less than about 500 nm,
for at least a four week period at ambient temperature.

3. The pharmaceutical composition according to claim 2 wherein the concentration of the at least one surface stabilizer is sufficient to maintain a particle size distribution of:
(a) a mean particle size of less than about 200 nm; and
(b) 90% of the particles with a particle size of less than about 300 nm,
for at least a four week period at ambient temperature.

4. The pharmaceutical composition according to claim 1 wherein the cyclooxygenase-2 selective inhibitor is selected from the group consisting of: rofecoxib, etoricoxib, celecoxib, valdecoxib and lumiracoxib.

5. The pharmaceutical composition according to claim 4 wherein the cyclooxygenase-2 selective inhibitor is rofecoxib.

6. (canceled)

7. (canceled)

8. The pharmaceutical composition according to claim 1 wherein the surface stabilizer is selected from the group consisting of: hydroxypropyl cellulose, hydroxypropyl cellulose super low viscosity, hydroxypropyl cellulose-low viscosity, hydroxypropyl methyl cellulose, methyl cellulose, hydroxyethyl cellulose, carboxymethyl cellulose sodium, polyvinylpyrrolidone (PVP), polyvinyl alcohol (PVA), poloxamer, tyloxapol, polyoxyethylene sorbitan fatty acid esters and polyethylene glycol.

9. The pharmaceutical composition according to claim 8 wherein the surface stabilizers are selected from the group consisting of: hydroxypropylmethylcellulose and poloxamer.

10. The pharmaceutical composition according to claim 9 wherein hydroxypropylmethylcellulose is selected from HPMC E3 and HPMC K3, and poloxamer is POLOXAMER 407.

11. The pharmaceutical composition according to claim 10 wherein the surface stabilizers are hydroxypropylmethyl cellulose K3 and POLOXAMER 407, wherein hydroxypropylmethylcellulose K3 is present in a concentration of about 0.01 to about 200 mg/mL and POLOXAMER 407 is present in a concentration of about 0.001 to about 200 mg/mL.

12. The pharmaceutical composition according to claim 11, wherein hydroxypropylmethylcellulose K3 is present in a concentration of about 0.1 to about 100 mg/mL and POLOXAMER 407 is present in a concentration of about 0.01 to about 50 mg/mL.

13. The pharmaceutical composition according to claim 12, wherein hydroxypropylmethylcellulose K3 is present in a concentration of about 1 to about 50 mg/mL and POLOXAMER 407 is present in a concentration of about 0.1 to about 10 mg/mL.

14. The pharmaceutical composition according to claim 10 wherein the surface stabilizers are hydroxypropylmethyl cellulose E3 and POLOXAMER 407, wherein hydroxypropylmethylcellulose E3 is present in a concentration of about 0.01 to about 200 mg/mL and POLOXAMER 407 is present in a concentration of about 0.001 to about 200 mg/mL.

15. The pharmaceutical composition according to claim 14 wherein hydroxypropylmethylcellulose E3 is present in a concentration of about 0.1 to about 100 mg/mL and POLOXAMER 407 is present in a concentration of about 0.01 to about 50 mg/mL.

16. The pharmaceutical composition according to claim 15 wherein hydroxypropylmethylcellulose E3 is present in a concentration of about 1 to about 50 mg/mL and POLOXAMER 407 is present in a concentration of about 0.1 to about 10 mg/mL.

17. The pharmaceutical composition according to claim 1 wherein the cyclooxygenase-2 selective inhibitor is rofecoxib present in a concentration of about 0.01 to about 600 mg/mL.

18. The pharmaceutical composition according to claim 17 wherein rofecoxib is present in a concentration of about 1 to about 300 mg/mL.

19. The pharmaceutical composition according to claim 18 wherein rofecoxib is present in a concentration of about 10 to about 100 mg/mL.

20. The pharmaceutical composition according to claim 2 having an initial particle size distribution of: a mean particle size of less than about 400 nm and 90% of the particles with a particle size less than about 500 nm.

21. The pharmaceutical composition according to claim 20 having an initial particle size distribution of: a mean particle size of less than about 200 nm and 90% of the particles with a particle size less than about 300 nm.

22. The pharmaceutical composition according to claim 21 having an initial particle size distribution of: a mean particle size of less than about 100 nm and 90% of the particles with a particle size less than about 150 nm.

23. The pharmaceutical composition according to claim 1 wherein the liquid dispersion medium is water.

24. The pharmaceutical composition according to claim 1 wherein the liquid dispersion medium is an isotonic agent.

25. The pharmaceutical composition according to claim 24 wherein the isotonic agent is selected from the group consisting of: NaCl (aq) and sugar (aq).

26.-39. (canceled)
